SEQUENCE LISTING (7 sequences)

SEQ ID NO: 1 - DNA, 3168 bp, Streptococcus
atgacaaaaaaacatcttaaaacgcttgccttggcacttactacagtatcagtagtgacatacagccaggaggtatatggattagaaagagaggaatcggtcaaacaagaacaaacccagtcagcttcagaagatgattggttcgaagaagataatgagaggaaaacaaatgtttctaaagagaattctactgttgatgaaacagttagtgatttattttctgatggaaatagtaataactctagttctaaaaccgagtcagtggtaagtgaccctaaacaagtccccaaagcaaaaccagaggttacacaagaagcaagcaattctagtaatgatgctagcaaagtagaagtaccaaaacaggatacagcttcaaaaaaggaaactctagaaacatcaacttgggaggcaaaagatttcgtaactagaggggatactttagtaggtttttcaaaatctggaattaataagttatctcaaacatcacacttggttttaccaagtcatgcagcagatggaactcaattgacacaagtagctagctttgcttttactccagataaaaagacggccattgcagaatatacaagtaggctaggagaaaatgggaaaccgagtcgtttagatattgatcagaaggaaattattgatgagggagaaatatttaatgcttaccagttgactaagcttactattccaaatggttataagtctattggtcaagatgcttttgtggacaataagaatattgctgaggttaaccttcctgagagtctcgagactatttcagactatgcttttgctcacatgtctttaaaacaagtaaagttaccagataacctaaaggtcattggagaattagctttttttgataatcagattggtggtaagctttacttgccacgtcacttgataaaattagcagaacgcgctttcaaatctaatcgtattcaaacagttgaatttttgggaagtaagcttaaggttataggagaagcaagttttcaagataataatctgaggaatgttatgcttccggatggacttgaaaaaatagaatcagaagcttttacaggaaatccaggagatgaacattacaacaatcaggttgtattgcgcacaaggacaggccaaaatccacatcaacttgcgactgagaatacttacgtcaatccggacaaatcattgtggcgtgcaacacctgatatggattataccaaatggttagaggaagattttacctatcaaaaaaatagtgttacaggtttttcaaataaaggcttacaaaaggtaagacgtaataaaaacttagaaattccaaaacaacacaatggtattactattactgaaattggtgataacgcttttcgcaatgttgattttcaaagtaaaactttacgtaaatatgatttggaagaaataaagctcccctcaactattcggaaaataggtgcttttgcttttcaatctaataacttgaaatcctttgaagcaagtgaagatttagaagagattaaagagggagcctttatgaataatcgtattggaactctagacttgaaagacaaacttatcaaaataggtgatgctgctttccatattaatcatatttatgccattgttcttccagaatctgtacaagaaataggacgttcagcttttcgacaaaatggtgcgcttcaccttatgtttatcggaaataaggttaaaacaattggtgaaatggcttttttatccaataaactggaaagtgtaaatctctctgagcaaaaacaattaaagacaattgaggtccaagctttttcggataatgcccttagtgaagtagtcttaccgccaaatttacagactattcgtgaagaggctttcaaaaggaatcatttgaaagaagtgaagggttcatctacattatctcagattacttttaatgcttttgatcaaaatgatggggacaaacgctttggtaagaaagtggttgttaggacacataataattctcatatgttagcagatggtgagcgttttatcattgatccagataagctatcttctacaatggtagaccttgaaaaggttttaaaaataatcgaaggtttagattactctacattacgtcagactactcaaactcagtttagagaaatgactactgcaggtaaagcgttgttatcaaaatctaacctccgacaaggagaaaaacaaaaattccttcaagaagcacaatttttccttggtcgcgttgatttggataaagccatagctaaagctgagaaggctttagtgaccaagaaggcaacaaagaatggtcatttgcttgagaggagtattaacaaagcggtattagcttataataatagtgctattaaaaaagctaatgttaagcgcttggaaaaagagttagacttgctgacagatttagtcgagggaaaaggaccattagcgcaagctacaatggtacaaggagtttatttattaaagacgcctttaccattgccagaatattatatcggattgaacgtttattttgacaagtctggaaaattgatttatgcacttgatatgagtgatactattggcgagggacaaaaagatgcatatggtaatcctatattaaatgttgacgaggataatgaaggttatcataccttggcagttgccactttagctgattatgaaggtctttatattaaagatattttaaatagttcccttgataagattaaagcaatacgccagattcctttggcaaaatatcatagattaggaattttccaagctatccgaaatgcagcggcagaagcagaccgattgcttcctaagacacctaaggggtacctaaatgaagtcccaaattatcgtaaaaaacaaatggagaaaaatttaaaaccagttgattataaaacgccgatttttaataaggctttacctaatgaaaaggtagacggtgatagagcggctaaaggtcataatataaatgcggagactaataattctgtagctgtaacaccaataaggtccgagcagcaattacataagtcacagtctgatgtaaatttacctcaaacaagttctaaaaataattttatatacgagattctaggatacgttagtttatgtttgcttttcctagtaactgctgggaaaaaaggaaaacgagcaagaaaataa

SEQ ID NO: 2 - protein, 1055 aa, Streptococcus
MTKKHLKTLALALTTVSVVTYSQEVYGLEREESVKQEQTQSASEDDWFEEDNERKTNVSKENSTVDETVSDLFSDGNSNNSSSKTESVVSDPKQVPKAKPEVTQEASNSSNDASKVEVPKQDTASKKETLETSTWEAKDFVTRGDTLVGFSKSGINKLSQTSHLVLPSHAADGTQLTQVASFAFTPDKKTAIAEYTSRLGENGKPSRLDIDQKEIIDEGEIFNAYQLTKLTIPNGYKSIGQDAFVDNKNIAEVNLPESLETISDYAFAHMSLKQVKLPDNLKVIGELAFFDNQIGGKLYLPRHLIKLAERAFKSNRIQTVEFLGSKLKVIGEASFQDNNLRNVMLPDGLEKIESEAFTGNPGDEHYNNQVVLRTRTGQNPHQLATENTYVNPDKSLWRATPDMDYTKWLEEDFTYQKNSVTGFSNKGLQKVRRNKNLEIPKQHNGITITEIGDNAFRNVDFQSKTLRKYDLEEIKLPSTIRKIGAFAFQSNNLKSFEASEDLEEIKEGAFMNNRIGTLDLKDKLIKIGDAAFHINHIYAIVLPESVQEIGRSAFRQNGALHLMFIGNKVKTIGEMAFLSNKLESVNLSEQKQLKTIEVQAFSDNALSEVVLPPNLQTIREEAFKRNHLKEVKGSSTLSQITFNAFDQNDGDKRFGKKVVVRTHNNSHMLADGERFIIDPDKLSSTMVDLEKVLKIIEGLDYSTLRQTTQTQFREMTTAGKALLSKSNLRQGEKQKFLQEAQFFLGRVDLDKAIAKAEKALVTKKATKNGHLLERSINKAVLAYNNSAIKKANVKRLEKELDLLTDLVEGKGPLAQATMVQGVYLLKTPLPLPEYYIGLNVYFDKSGKLIYALDMSDTIGEGQKDAYGNPILNVDEDNEGYHTLAVATLADYEGLYIKDILNSSLDKIKAIRQIPLAKYHRLGIFQAIRNAAAEADRLLPKTPKGYLNEVPNYRKKQMEKNLKPVDYKTPIFNKALPNEKVDGDRAAKGHNINAETNNSVAVTPIRSEQQLHKSQSDVNLPQTSSKNNFIYEILGYVSLCLLFLVTAGKKGKRARK

SEQ ID NO: 3 - DNA, 34 bp, Artificial
  primer
gcgcccatggtgcaggaggtatatggttagaaag

SEQ ID NO: 4 - DNA, 30 bp, Artificial
  primer
ggtggatccgagaaaggctttattgtaatg

SEQ ID NO: 5 - DNA, 36 bp, Artificial
  primer
catattaattgcggccgcttttcttgctcgttttcc

SEQ ID NO: 6 - DNA, 34 bp, Artificial
  primer
cgttggatcctcaggaggtatatggattagaaag

SEQ ID NO: 7 - DNA, 31 bp, Artificial
  primer
catcgtcgacttattttcttgctcgttttcc

(19) United States
(12) Patent Application Publication
(10) Pub. No.: US 2004/0171113 A1
(43) Pub. Date: Sep. 2, 2004

(54) Antigens of group b streptococcus and corresponding dna fragments

(76) Inventors: Denis Martin, St-Augustin-de-Desmaures (CA); Josee Hamel, Sillery (CA); Bernard R Brodeur, Sillery (CA); Stephane Rioux, Beauport (CA); Martine Boyer, Ste-Foy (CA)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/476,614

(22) PCT Filed: May 2, 2002
(86) PCT No.: PCT/CA02/00664

(30) Foreign Application Priority Data
May 2, 2001 (US) 60287712

(51) Int. Cl. C07H 21/04; C12N 1/21; C07K 14/315

(52) U.S. Cl. 435/69.1; 435/320.1; 435/252.3; 530/350; 536/23.7

(57) ABSTRACT

The present invention relates to antigens, more particularly antigens of Group B Streptococcus (GBS) (S. agalactiae) which may be useful to prevent, diagnose and/or treat streptococcal infections.

### FIELD OF THE INVENTION

The present invention is related to antigens, more particularly polypeptides of Group B Streptococcus (GBS) (S. agalactiae) which may be used to prevent, diagnose, and/or treat GBS infections.

### BACKGROUND OF THE INVENTION.

Streptococcus are gram (+) bacteria that are differentiated by group specific carbohydrate antigens A through O found on their cell surface. Streptococcus groups are further distinguished by type-specific capsular polysaccharide antigens. Several serotypes have been identified for the GBS: Ia, Ib, II, III, IV, V, VI, VII and VIII. GBS also contains antigenic proteins known as “C-proteins” (alpha, beta, gamma and delta), some of which have been cloned.

Although GBS is a common component of the normal human vaginal and colonic flora this pathogen has long been recognized as a major cause of infections in neonates or infants, expectant mothers, some non-pregnant adults as well as mastitis in dairy herds. Expectant mothers exposed to GBS are at risk of postpartum infection and may transfer the infection to their baby as the child passes through the birth canal.

GBS infections in infants are restricted to very early infancy. Approximately 80% of infant infections occur in the first days of life, so-called early-onset disease. Late-onset infections occur in infants between 1 week and 2 to 3 months of age. Clinical syndromes of GBS disease in newborns include sepsis, meningitis, pneumonia, cellulitis, osteomyelitis, septic arthritis, endocarditis and epiglottis. In addition to acute illness due to GBS, which is itself costly, GBS infections in newborns can result in death, disability, and, in rare instances, recurrence of infection. Although the organism is sensitive to antibiotics, the high attack rate and rapid onset of sepsis in neonates and meningitis in infants results in high morbidity and mortality.

Among pregnant women, GBS causes clinical illness ranging from mild urinary tract infection to life-threatening sepsis and meningitis, including also osteomyelitis, endocarditis, amniotis, endometritis, wound infections (postcesarean and postepisiotomy), cellulitis, fasciitis.

Among non-pregnant adults, the clinical presentations of invasive GBS disease most often take the form of primary bacteremia but also skin or soft tissue infection, pneumonia, urosepsis, endocarditisi, peritonitis, meningitis, empyema. Skin or soft tissue infections include cellulitis, infected peripheral ulcers, osteomyelitis, septic arthritis and decubiti or wound infections. Among people at risk, there are debilitated hosts such as people with a chronic disease such as diabetes mellitus and cancer, or elderly people.

GBS infections can also occur in animals and cause mastitis in dairy herds.

Type-specific polysaccharides have proven to be poorly immunogenic in hosts and are restricted to the particular serotype from which the polysaccharide originates. Further, capsular polysaccharide elicit a T cell independent response i.e. no IgG production. Consequently capsular polysaccharide antigens are unsuitable as a vaccine component for protection against GBS infection.

Others have focused on the C-protein beta antigen which demonstrated immunogenic properties in mice and rabbit models. This protein was found to be unsuitable as a human vaccine because of its undesirable property of interacting with high affinity and in a non-immunogenic manner with the Fc region of human IgA. The C-protein alpha antigen is rare in type III serotypes of GBS which is the serotype responsible for most GBS mediated conditions and is therefore of little use as a vaccine component.

There remains an unmet need for GBS antigens that may be used as vaccine components for the prophylaxis and/or therapy of GBS infection.

### SUMMARY OF THE INVENTION

According to one aspect, the present invention provides an isolated polynucleotide encoding a polypeptide having at least 80% identity to a second polypeptide comprising a sequence chosen from SEQ ID NOS: 2, or fragments or analogs thereof.

According to one aspect, the present invention relates to polypeptides comprising SEQ ID NO: 2 or fragments or analogs thereof.

In other aspects, there are provided polypeptides encoded by polynucleotides of the invention, pharmaceutical composition, vectors comprising polynucleotides of the invention operably linked to an expression control region, as well as host cells transfected with said vectors and processes for producing polypeptides comprising culturing said host cells under conditions suitable for expression.

### BRIEF DESCRIPTION OF THE DRAWINGS

FIG. 1 represents the DNA sequence of BVH-A4 gene from serotype III Group B streptococcus strain COH1; (SEQ ID NO: 1). The underlined portion of the sequence represents the region coding for the leader peptide.

FIG. 2 represents the amino acid sequence of BVH-A4 protein from serotype III Group B streptococcus strain COH1; (SEQ ID NO: 2). The underlined sequence represents the 22 amino acid residues leader peptide.

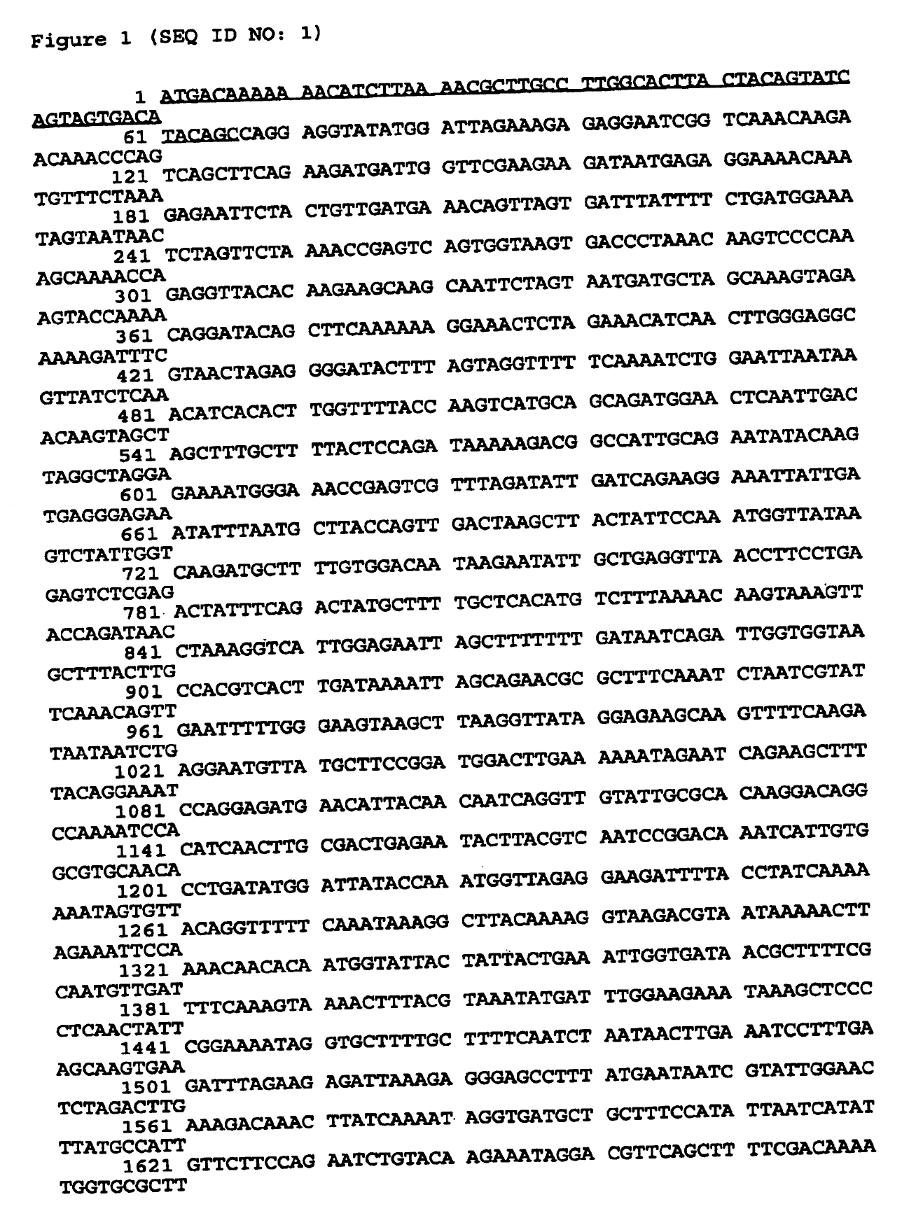

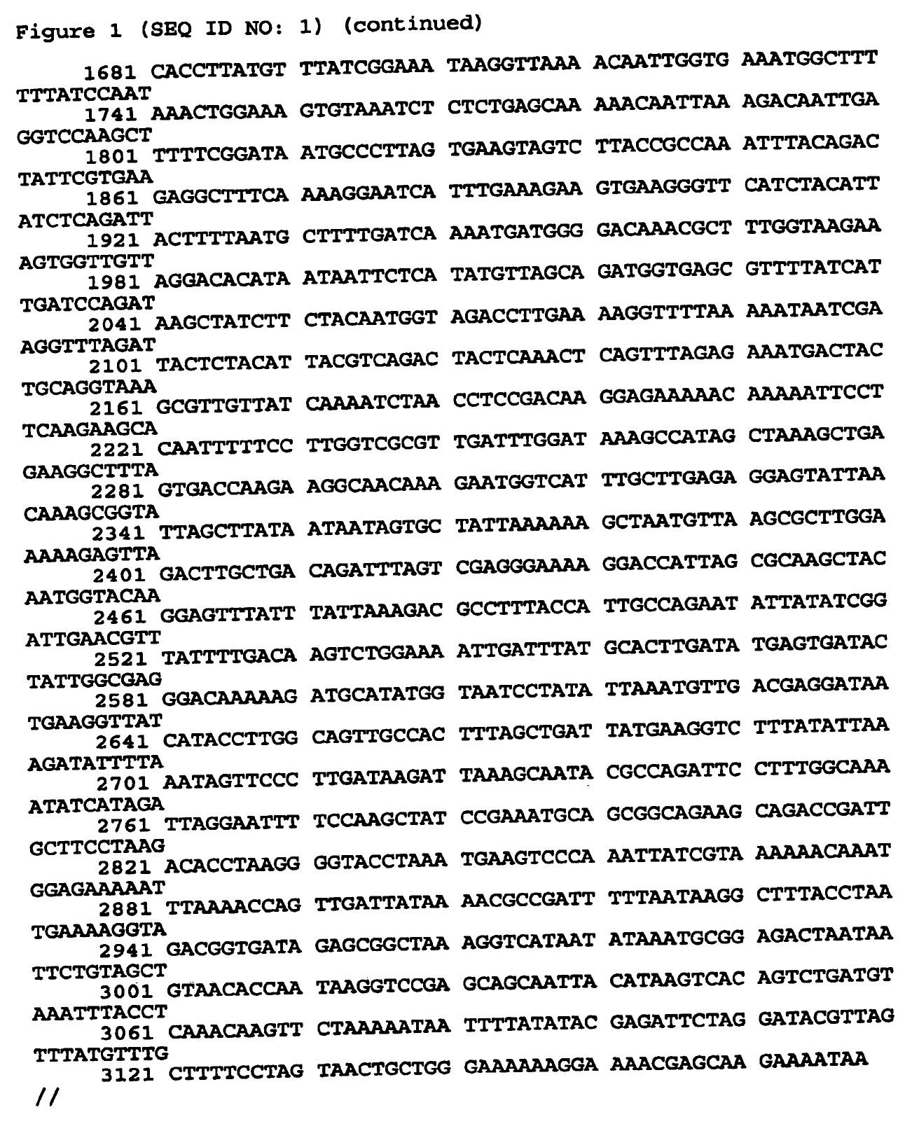

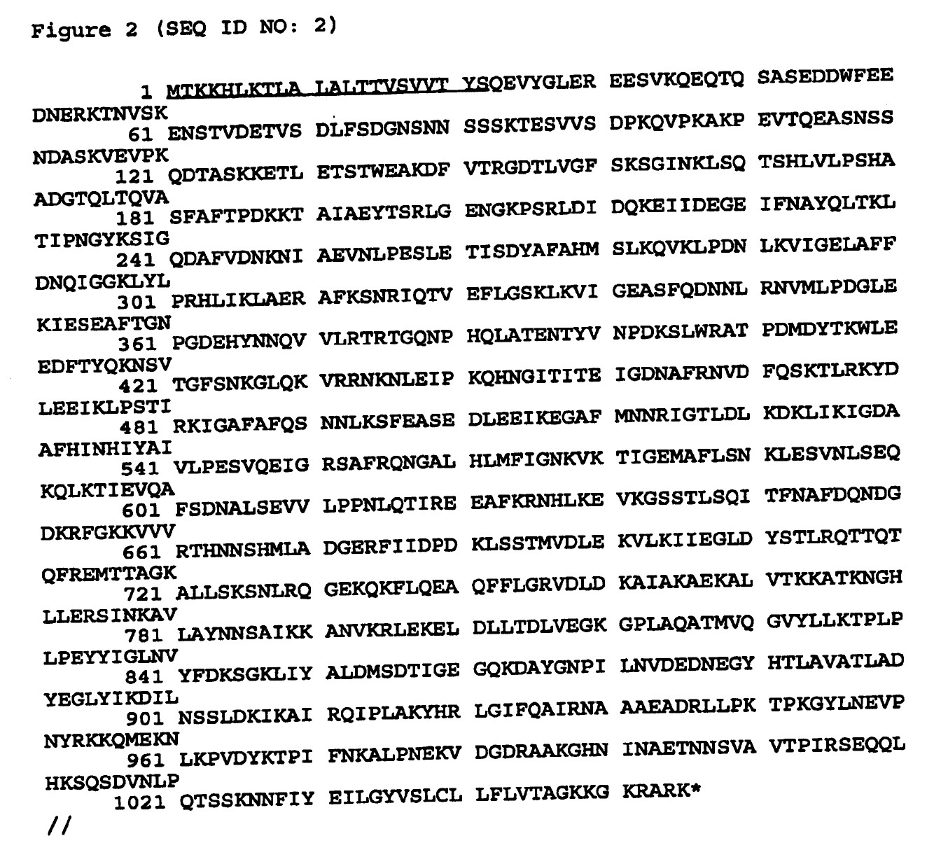

### DETAILED DESCRIPTION OF THE INVENTION

The present invention provides purified and isolated polynucleotides, which encode Streptococcal polypeptides that may be used to prevent, diagnose and/or treat Streptococcal infection.

According to one aspect, the present invention provides an isolated polynucleotide encoding a polypeptide having at least 80% identity to a second polypeptide comprising a sequence chosen from SEQ ID NO: 2 or fragments or analogs thereof.

According to one aspect, the present invention provides an isolated polynucleotide encoding a polypeptide having at least 90% identity to a second polypeptide comprising a sequence chosen from SEQ ID NO: 2 or fragments or analogs thereof.

According to one aspect, the present invention provides an isolated polynucleotide encoding a polypeptide having at least 95% identity to a second polypeptide comprising a sequence chosen from SEQ ID NO: 2 or fragments or analogs thereof.

According to one aspect, the present invention relates to polypeptides comprising SEQ ID NO: 2 or fragments or analogs thereof.

According to one aspect, the present invention provides a polynucleotide encoding an epitope bearing portion of a polypeptide comprising SEQ ID NO: 2 or fragments or analogs thereof.

According to one aspect, the present invention relates to epitope bearing portions of a polypeptide comprising SEQ ID NO: 2 or fragments or analogs thereof.

According to one aspect, the present invention provides an isolated polynucleotide encoding a polypeptide having at least 80% identity to a second polypeptide comprising SEQ ID NO: 2.

According to one aspect, the present invention provides an isolated polynucleotide encoding a polypeptide having at least 90% identity to a second polypeptide comprising SEQ ID NO: 2.

According to one aspect, the present invention provides an isolated polynucleotide encoding a polypeptide having at least 95% identity to a second polypeptide comprising SEQ ID NO: 2.

According to one aspect, the present invention relates to polypeptides comprising SEQ ID NO: 2.

According to one aspect, the present invention provides a polynucleotide encoding an epitope bearing portion of a polypeptide comprising SEQ ID NO: 2.

According to one aspect, the present invention relates to epitope bearing portions of a polypeptide comprising SEQ ID NO: 2.

According to one aspect, the present invention provides an isolated polynucleotide comprising a polynucleotide chosen from:

(a) a polynucleotide encoding a polypeptide having at least 80% identity to a second polypeptide comprising a sequence chosen from: SEQ ID NO: 2 or fragments or analogs thereof;

(b) a polynucleotide encoding a polypeptide having at least 95% identity to a second polypeptide comprising a sequence chosen from: SEQ ID NO: 2 or fragments or analogs thereof;

(c) a polynucleotide encoding a polypeptide comprising a sequence chosen from: SEQ ID NO: 2 or fragments or analogs thereof;

(d) a polynucleotide encoding a polypeptide capable of generating antibodies having binding specificity for a polypeptide comprising a sequence chosen from: SEQ ID NO: 2 or fragments or analogs thereof;

(e) a polynucleotide encoding an epitope bearing portion of a polypeptide comprising a sequence chosen from SEQ ID NO: 2 or fragments or analogs thereof;

(f) a polynucleotide comprising a sequence chosen from SEQ ID NO: 1 or fragments or analogs thereof;

(g) a polynucleotide that is complementary to a polynucleotide in (a), (b), (c), (d), (e) or (f).

According to one aspect, the present invention provides an isolated polynucleotide comprising a polynucleotide chosen from:

(a) a polynucleotide encoding a polypeptide having at least 80% identity to a second polypeptide comprising a sequence chosen from: SEQ ID NO: 2;

(b) a polynucleotide encoding a polypeptide having at least 95% identity to a second polypeptide comprising a sequence chosen from: SEQ ID NO: 2;

(c) a polynucleotide encoding a polypeptide comprising a sequence chosen from: SEQ ID NO: 2;

(d) a polynucleotide encoding a polypeptide capable of raising antibodies having binding specificity for a polypeptide comprising a sequence chosen from: SEQ ID NO: 2;

(e) a polynucleotide encoding an epitope bearing portion of a polypeptide comprising a sequence chosen from SEQ ID NO: 2;

(f) a polynucleotide comprising a sequence chosen from SEQ ID NO: 1;

(g) a polynucleotide that is complementary to a polynucleotide in (a), (b), (c), (d), (e) or (f).

According to one aspect, the present invention provides an isolated polypeptide comprising a polypeptide chosen from:

(a) a polypeptide having at least 80% identity to a second polypeptide comprising SEQ ID NO: 2, or fragments or analogs thereof;

(b) a polypeptide having at least 95% identity to a second polypeptide comprising SEQ ID NO: 2, or fragments or analogs thereof;

(c) a polypeptide comprising SEQ ID NO: 2, or fragments or analogs thereof;

(d) a polypeptide capable of raising antibodies having binding specificity for a polypeptide comprising SEQ ID NO: 2, or fragments or analogs thereof;

(e) an epitope bearing portion of a polypeptide comprising SEQ ID NO: 2, or fragments or analogs thereof;

(f) the polypeptide of (a), (b), (c), (d), (e) or (f) wherein the N-terminal Met residue is deleted;

(g) the polypeptide of (a), (b), (c), (d), (e) or (f) wherein the secretory amino acid sequence is deleted.

According to one aspect, the present invention provides an isolated polypeptide comprising a polypeptide chosen from:

(a) a polypeptide having at least 80% identity to a second polypeptide comprising SEQ ID NO: 2;

(b) a polypeptide having at least 95% identity to a second polypeptide comprising SEQ ID NO: 2;

(c) a polypeptide comprising SEQ ID NO: 2;

(d) a polypeptide capable of raising antibodies having binding specificity for a polypeptide comprising SEQ ID NO: 2;

(e) an epitope bearing portion of a polypeptide comprising SEQ ID NO: 2;

(f) the polypeptide,of (a), (b), (c), (d), (e) or (f) wherein the N-terminal Met residue is deleted;

(g) the polypeptide of (a), (b), (c), (d), (e) or (f) wherein the secretory amino acid sequence is deleted.

Those skilled in the art will appreciate that the invention includes DNA molecules, i.e. polynucleotides and their complementary sequences that encode analogs such as mutants, variants, homologues and derivatives of such polypeptides, as described herein in the present patent application. The invention also includes RNA molecules corresponding to the DNA molecules of the invention. In addition to the DNA and RNA molecules, the invention includes the corresponding polypeptides and monospecific antibodies that specifically bind to such polypeptides.

In a further embodiment, the polypeptides in accordance with the present invention are antigenic.

In a further embodiment, the polypeptides in accordance with the present invention are immunogenic.

In a further embodiment, the polypeptides in accordance with the present invention can elicit an immune response in a host.

In a further embodiment, the present invention also relates to polypeptides which are able to raise antibodies having binding specificity to the polypeptides of the present invention as defined above.

An antibody that “has binding specificity” is an antibody that recognizes and binds the selected polypeptide but which does not substantially recognize and bind other molecules in a sample, e.g., a biological sample, which naturally includes the selected peptide. Specific binding can be measured using an ELISA assay in which the selected polypeptide is used as an antigen.

In accordance with the present invention, “protection” in the biological studies is defined by a significant increase in the survival curve, rate or period. Statistical analysis using the Log rank test to compare survival curves, and Fisher exact test to compare survival rates and numbers of days to death, respectively, might be useful to calculate P values and determine whether the difference between the two groups is statistically significant. P values of 0.05 are regarded as not significant.

In an additional aspect of the invention there are provided antigenic/immunogenic fragments of the polypeptides of the invention, or of analogs thereof.

The fragments of the present invention should include one or more such epitopic regions or be sufficiently similar to such regions to retain their antigenic/immunogenic properties. Thus, for fragments according to the present invention the degree of identity is perhaps irrelevant, since they may be 100% identical to a particular part of a polypeptide or analog thereof as described herein. The present invention further provides fragments having at least 10 contiguous amino acid residues from the polypeptide sequences of the present invention. In one embodiment, at least 15 contiguous amino acid residues. In one embodiment, at least 20 contiguous amino acid residues.

The skilled person will appreciate that analogs of the polypeptides of the invention will also find use in the context of the present invention, i.e. as antigenic/immunogenic material. Thus, for instance proteins or polypeptides which include one or more additions, deletions, substitutions or the like are encompassed by the present invention.

As used herein, “fragments”, “analogs” or “derivatives” of the polypeptides of the invention include those polypeptides in which one or more of the amino acid residues are substituted with a conserved or non-conserved amino acid residue (preferably conserved) and which may be natural or unnatural. In one embodiment, derivatives and analogs of polypeptides of the invention will have about 80% identity with those sequences illustrated in the figures or fragments thereof. That is, 80% of the residues are the same. In a further embodiment, polypeptides will have greater than 80% identity. In a further embodiment, polypeptides will have greater than 85% identity. In a further embodiment, polypeptides will have greater than 90% identity. In a further embodiment, polypeptides will have greater than 95% identity. In a further embodiment, polypeptides will have greater than 99% identity. In a further embodiment, analogs of polypeptides of the invention will have fewer than about 20 amino acid residue substitutions, modifications or deletions and more preferably less than 10.

These substitutions are those having a minimal influence on the secondary structure and hydropathic nature of the polypeptide. Preferred substitutions are those known in the art as conserved, i.e. the substituted residues share physical or chemical properties such as hydrophobicity, size, charge or functional groups. These include substitutions such as those described by Dayhoff, M. in Atlas of Protein Sequence and Structure 5, 1978 and by Argos, P. in EMBO J. 8, 779-785, 1989. For example, amino acids, either natural or unnatural, belonging to one of the following groups represent conservative changes:

ala, pro, gly, gln, asn, ser, thr, val;
cys, ser, tyr, thr;
val, ile, leu, met, ala, phe;
lys, arg, orn, his;
and phe, tyr, trp, his.

The preferred substitutions also include substitutions of D-enantiomers for the corresponding L-amino acids.

In an alternative approach, the analogs could be fusion proteins, incorporating moieties which render purification easier, for example by effectively tagging the desired polypeptide. It may be necessary to remove the “tag” or it may be the case that the fusion polypeptide itself retains sufficient antigenicity to be useful.

The percentage of homology is defined as the sum of the percentage of identity plus the percentage of similarity or conservation of amino acid type.

In one embodiment, analogs of polypeptides of the invention will have about 80% identity with those sequences illustrated in the figures or fragments thereof. That is, 80% of the residues are the same. In a further embodiment, polypeptides will have greater than 85% identity. In a further embodiment, polypeptides will have greater than 90% identity. In a further embodiment, polypeptides will have greater than 95% identity. In a further embodiment, polypeptides will have greater than 99% identity. In a further embodiment, analogs of polypeptides of the invention will have fewer than about 20 amino acid residue substitutions, modifications or deletions and more preferably less than 10.

In one embodiment, analogs of polypeptides of the invention will have about 80% homology with those sequences illustrated in the figures or fragments thereof. In a further embodiment, polypeptides will have greater than 85% homology. In a further embodiment, polypeptides will have greater than 90% homology. In a further embodiment, polypeptides will have greater than 95% homology. In a further embodiment, polypeptides will have greater than 99% homology. In a further embodiment, analogs of polypeptides of the invention will have fewer than about 20 amino acid residue substitutions, modifications or deletions and more preferably less than 10.

One can use a program such as the CLUSTAL™ program to compare amino acid sequences. This program compares amino acid sequences and finds the optimal alignment by inserting spaces in either sequence as appropriate. It is possible to calculate amino acid identity or homology for an optimal alignment. A program like BLASTx™ will align the longest stretch of similar sequences and assign a value to the fit. It is thus possible to obtain a comparison where several regions of similarity are found, each having a different score. Both types of identity analysis are contemplated in the present invention.

In an alternative approach, the analogs or derivatives could be fusion polypeptides, incorporating moieties which render purification easier, for example by effectively tagging the desired protein or polypeptide, it may be necessary to remove the “tag” or it may be the case that the fusion polypeptide itself retains sufficient antigenicity to be useful.

It is well known that is possible to screen an antigenic polypeptide to identify epitopic regions, i.e. those regions which are responsible for the polypeptide's antigenicity or immunogenicity. Methods for carrying out such screening are well known in the art. Thus, the fragments of the present invention should include one or more such epitopic regions or be sufficiently similar to such regions to retain their antigenic/immunogenic properties.

Thus, for fragments according to the present invention the degree of identity is perhaps irrelevant, since they may be 100% identical to a particular part of a polypeptide, analog as described herein.

Thus, what is important for analogs, derivatives and fragments is that they possess at least a degree of the antigenicity/immunogenicity of the protein or polypeptide from which they are derived.

Also included are polypeptides which have fused thereto other compounds which alter the polypeptides biological or pharmacological properties i.e. polyethylene glycol (PEG) to increase half-life; leader or secretory amino acid sequences for ease of purification; prepro- and pro-sequences; and (poly)saccharides.

Furthermore, in those situations where amino acid regions are found to be polymorphic, it may be desirable to vary one or more particular amino acids to more effectively mimic the different epitopes of the different Streptococcus strains.

Moreover, the polypeptides of the present invention can be modified by terminal —NH₂ acylation (eg. by acetylation, or thioglycolic acid amidation, terminal carboxy amidation, e.g. with ammonia or methylamine) to provide stability, increased hydrophobicity for linking or binding to a support or other molecule.

Also contemplated are hetero and homo polypeptide multimers of the polypeptide fragments and analogues. These polymeric forms include, for example, one or more polypeptides that have been cross-linked with cross-linkers such as avidin/biotin, gluteraldehyde or dimethyl-superimidate. Such polymeric forms also include polypeptides containing two or more tandem or inverted contiguous sequences, produced from multicistronic mRNAs generated by recombinant DNA technology.

In a further embodiment, the present invention also relates to chimeric polypeptides which comprise one or more polypeptides or fragments or analogs thereof as defined in the figures of the present application.

In a further embodiment, the present invention also relates to chimeric polypeptides comprising two or more polypeptides having a sequence chosen from SEQ ID NO: 2, or fragments or analogs thereof; provided that the polypeptides are linked as to formed a chimeric polypeptide.

In a further embodiment, the present invention also relates to chimeric polypeptides comprising two or more polypeptides having a sequence chosen from SEQ ID NO: 2 provided that the polypeptides are linked as to formed a chimeric polypeptide.

Preferably, a fragment, analog or derivative of a polypeptide of the invention will comprise at least one antigenic region i.e. at least one epitope.

In order to achieve the formation of antigenic polymers (i.e. synthetic multimers), polypeptides may be utilized having bishaloacetyl groups, nitroarylhalides, or the like, where the reagents being specific for thio groups. Therefore, the link between two mercapto groups of the different polypeptides may be a single bond or may be composed of a linking group of at least two, typically at least four, and not more than 16, but usually not more than about 14 carbon atoms.

In a particular embodiment, polypeptide fragments and analogs of the invention do not contain a starting residue, such as methionine (Met) or valine (Val). Preferably, polypeptides will not incorporate a leader or secretory sequence (signal sequence). The signal portion of a polypeptide of the invention may be determined according to established molecular biological techniques. In general, the polypeptide of interest may be isolated from a streptococcal culture and subsequently sequenced to determine the initial residue of the mature protein and therefore the sequence of the mature polypeptide.

It is understood that polypeptides can be produced and/or used without their start codon (methionine or valine) and/or without their leader peptide to favor production and purification of recombinant polypeptides. It is known that cloning genes without sequences encoding leader peptides will restrict the polypeptides to the cytoplasm of E. coli and will facilitate their recovery (Glick, B. R. and Pasternak, J. J. (1998) Manipulation of gene expression in prokaryotes. In “Molecular biotechnology: Principles and applications of recombinant DNA”, 2nd edition, ASM Press, Washington D.C., p.109-143).

According to another aspect of the invention, there are also provided (i) a composition of matter containing a polypeptide of the invention, together with a carrier, diluent or adjuvant; (ii) a pharmaceutical composition comprising a polypeptide of the invention and a carrier, diluent or adjuvant; (iii) a vaccine comprising a polypeptide of the invention and a carrier, diluent or adjuvant; (iv) a method for inducing an immune response against Streptococcus, in a host, by administering to the host, an immunogenically effective amount of a polypeptide of the invention to elicit an immune response, e.g., a protective immune response to Streptococcus; and particularly., (v) a method for preventing and/or treating a Streptococcus infection, by administering a prophylactic or therapeutic amount of a polypeptide of the invention to a host in need.

According to another aspect of the invention, there are also provided (i) a composition of matter containing a polynucleotide of the invention, together with a carrier, diluent or adjuvant; (ii) a pharmaceutical composition comprising a polynucleotide of the invention and a carrier, diluent or adjuvant; (iii) a method for inducing an immune response against Streptococcus, in a host, by administering to the host, an immunogenically effective amount of a polynucleotide of the invention to elicit an immune response, e.g., a protective immune response to Streptococcus; and particularly, (iv) a method for preventing and/or treating a Streptococcus infection, by administering a prophylactic or therapeutic amount of a polynucleotide of the invention to a host in need.

Before immunization, the polypeptides of the invention can also be coupled or conjugated to carrier proteins such as tetanus toxin, diphtheria toxin, hepatitis B virus surface antigen, poliomyelitis virus VP1 antigen or any other viral or bacterial toxin or antigen or any suitable proteins to stimulate the development of a stronger immune response. This coupling or conjugation can be done chemically or genetically. A more detailed description of peptide-carrier conjugation is available in Van Regenmortel, M. H. V., Briand J. P., Muller S., Plaué S., <<Synthetic Polypeptides as antigens>> in Laboratory Techniques in Biochemistry and Molecular Biology, Vol. 19 (ed.) Burdou, R. H. & Van Knippenberg P. H. (1988)., Elsevier N.Y.

According to another aspect, there are provided pharmaceutical compositions comprising one or more Streptococcal polypeptides of the invention in a mixture with a pharmaceutically acceptable adjuvant. Suitable adjuvants include (1) oil-in-water emulsion formulations such as MF59™, SAF™, Ribi™; (2) Freund's complete or incomplete adjuvant; (3) salts i.e. AlK(SO₄)₂, AlNa(SO₄)₂, AlNH₄(SO₄)₂, Al(OH)₃, AlPO₄, silica, kaolin; (4) saponin derivatives such as Stimulon™ or particles generated therefrom such as ISCOMs (immunostimulating complexes); (5) cytokines such as interleukins, interferons, macrophage colony stimulating factor (M-CSF), tumor necrosis factor (TNF); (6) other substances such as carbon polynucleotides i.e. poly IC and poly AU, detoxified cholera toxin (CTB) and E. coli heat labile toxin for induction of mucosal immunity. A more detailed description of adjuvant is available in a review by M. Z. I Khan et al. in Pharmaceutical Research, vol. 11, No. 1 (1994) pp2-11, and also in another review by Gupta et al., in Vaccine, Vol. 13, No. 14, pp1263-1276 (1995) and in WO 99/24578. Preferred adjuvants include QuilA™, QS21™, Alhydrogel™ and Adjuphos™.

Pharmaceutical compositions of the invention may be administered parenterally by injection, rapid infusion, nasopharyngeal absorption, dermoabsorption, or buccal or oral.

Pharmaceutical compositions of the invention are used for the prophylaxis or treatment of streptococcal infection and/or diseases and symptoms mediated by streptococcal infection as described in Manual of Clinical Microbiology, P. R. Murray (Ed, in chief), E. J. Baron, M. A. Pfaller, F. C. Tenover and R. H. Yolken. ASM Press, Washington, D.C. seventh edition, 1999, 1773p. In one embodiment, pharmaceutical compositions of the present invention are used for the prophylaxis or treatment of pharyngitis, erysipelas and impetigo, scarlet fever, and invasive diseases such as bacteremia and necrotizing fasciitis and also toxic shock. In one embodiment, pharmaceutical compositions of the invention are used for the treatment or prophylaxis of Streptococcus infection and/or diseases and symptoms mediated by Streptococcus infection, in particular group B Streptococcus (GBS or S. agalactiae), group A Streptococcus (Streptococcus pyogenes), S. pneumoniae, S. dysgalactiae, S. uberis, S. nocardia as well as Staphylococcus aureus. In a further embodiment, the Streptococcus infection is group B Streptococcus (GBS or S. agalactiae).

In a further embodiment, the invention provides a method for prophylaxis or treatment of Streptococcus infection in a host susceptible to Streptococcus infection comprising administering to said host a prophylactic or therapeutic amount of a composition of the invention.

In a further embodiment, the invention provides a method for prophylaxis or treatment of GBS infection in a host susceptible to GBS infection comprising administering to said host a prophylactic or therapeutic amount of a composition of the invention.

As used in the present application, the term “host” includes mammals. In a further embodiment, the mammal is a member of a dairy herd. In a further embodiment, the mammal is an expectant mother. In a further embodiment, the mammal is human. In a further embodiment, the host is a pregnant woman. In a further embodiment, the host is a non-pregnant adult. In a further embodiment, the host is a neonate or an infant.

In a particular embodiment, pharmaceutical compositions are administered to those hosts at risk of streptococcus infection such as infants, elderly and immunocompromised hosts.

Pharmaceutical compositions are preferably in unit dosage form of about 0.001 to 100 μg/kg (antigen/body weight) and more preferably 0.01 to 10 μg/kg and most preferably 0.1 to 1 μg/kg 1 to 3 times with an interval of about 1 to 6 week intervals between immunizations.

Pharmaceutical compositions are preferably in unit dosage form of about 0.1 μg to 10 mg and more preferably 1 μg to 1 mg and most preferably 10 to 100 μg 1 to 3 times with an interval of about 1 to 6 week intervals between immunizations.

According to another aspect, there are provided polynucleotides encoding polypeptides characterized by the amino acid sequence comprising SEQ ID NO: 2 or fragments or analogs thereof.

In one embodiment, polynucleotides are those illustrated in SEQ ID NO: 1 which may include the open reading frames (ORF), encoding the polypeptides of the invention.

It will be appreciated that the polynucleotide sequences illustrated in the figures may be altered with degenerate codons yet still encode the polypeptides of the invention. Accordingly the present invention further provides polynucleotides which hybridize to the polynucleotide sequences herein above described (or the complement sequences thereof) having 80% identity between sequences. In one embodiment, at least 85% identity between sequences. In one embodiment, at least 90% identity between sequences. In a further embodiment, polynucleotides are hybridizable under stringent conditions i.e. having at least 95% identity. In a further embodiment, more than 97% identity.

Suitable stringent conditions for hybridation can be readily determined by one of skilled in the art (see for example Sambrook et al., (1989) Molecular cloning: A Laboratory Manual, 2^{nd} ed, Cold Spring Harbor, N.Y.; Current Protocols in Molecular Biology, (1999) Edited by Ausubel F. M. et al., John Wiley & Sons, Inc., N.Y.).

In a further embodiment, the present invention provides polynucleotides that hybridize under stringent conditions to either

(a) a DNA sequence encoding a polypeptide or

(b) the complement of a DNA sequence encoding a polypeptide;

wherein said polypeptide comprises SEQ ID NO:2 or fragments or analogs thereof.

In a further embodiment, the present invention provides polynucleotides that hybridize under stringent conditions to either

(a) a DNA sequence encoding a polypeptide or

(b) the complement of a DNA sequence encoding a polypeptide;

wherein said polypeptide comprises SEQ ID NO: 2.

In a further embodiment, the present invention provides polynucleotides that hybridize under stringent conditions to either

(a) a DNA sequence encoding a polypeptide or

(b) the complement of a DNA sequence encoding a polypeptide;

wherein said polypeptide comprises at least 10 contiguous amino acid residues from a polypeptide comprising SEQ ID NO:2 or fragments or analogs thereof.

In a further embodiment, the present invention provides polynucleotides that hybridize under stringent conditions to either

(a) a DNA sequence encoding a polypeptide or

(b) the complement of a DNA sequence encoding a polypeptide;

wherein said polypeptide comprises at least 10 contiguous amino acid residues from a polypeptide comprising SEQ ID NO: 2.

In a further embodiment, polynucleotides of the invention are those encoding polypeptides of the invention illustrated in SEQ ID NO: 2 or fragments or analogs thereof.

In a further embodiment, polynucleotides of the invention are those illustrated in SEQ ID NO: 1 encoding polypeptides of the invention or fragments or analogs thereof.

In a further embodiment, polynucleotides of the invention are those encoding polypeptides of the invention illustrated in SEQ ID NO: 2.

In a further embodiment, polynucleotides of the invention are those illustrated in SEQ ID NO: 1 encoding polypeptides of the invention.

As will be readily appreciated by one skilled in the art, polynucleotides include both DNA and RNA.

The present invention also includes polynucleotides complementary to the polynucleotides described in the present application.

In a further aspect, polynucleotides encoding polypeptides of the invention, or fragments, analogs or derivatives thereof, may be used in a DNA immunization method. That is, they can be incorporated into a vector which is replicable and expressible upon injection thereby producing the antigenic polypeptide in vivo. For example polynucleotides may be incorporated into a plasmid vector under the control of the CMV promoter which is functional in eukaryotic cells. Preferably the vector is injected intramuscularly.

According to another aspect, there is provided a process for producing polypeptides of the invention by recombinant techniques by expressing a polynucleotide encoding said polypeptide in a host cell and recovering the expressed polypeptide product.

Alternatively, the polypeptides can be produced according to established synthetic chemical techniques i.e. solution phase or solid phase synthesis of oligopeptides which are ligated to produce the full polypeptide (block ligation).

General methods for obtention and evaluation of polynucleotides and polypeptides are described in the following references: Sambrook et al, Molecular Cloning: A Laboratory Manual, 2nd ed, Cold Spring Harbor, N.Y., 1989; Current Protocols in Molecular Biology, Edited by Ausubel F. M. et al., John Wiley and Sons, Inc. New York; PCR Cloning Protocols, from Molecular Cloning to Genetic Engineering, Edited by White B. A., Humana Press, Totowa, N.J., 1997, 490 pages; Protein Purification, Principles and Practices, Scopes R. K., Springer-Verlag, N.Y., 3rd Edition, 1993, 380 pages; Current Protocols in Immunology, Edited by Coligan J. E. et al., John Wiley & Sons Inc., New York.

The present invention provides a process for producing a polypeptide comprising culturing a host cell of the invention under conditions suitable for expression of said polypeptide.

For recombinant production, host cells are transfected with vectors which encode the polypeptides of the invention, and then cultured in a nutrient media modified as appropriate for activating promoters, selecting transformants or amplifying the genes. Suitable vectors are those that are viable and replicable in the chosen host and include chromosomal, non-chromosomal and synthetic DNA sequences e.g. bacterial plasmids, phage DNA, baculovirus, yeast plasmids, vectors derived from combinations of plasmids and phage DNA. The polypeptide sequence may be incorporated in the vector at the appropriate site using restriction enzymes such that it is operably linked to an expression control region comprising a promoter, ribosome binding site (consensus region or Shine-Dalgarno sequence), and optionally an operator (control element). One can select individual components of the expression control region that are appropriate for a given host and vector according to established molecular biology principles (Sambrook et al, Molecular Cloning: A Laboratory Manual, 2nd ed, Cold Spring Harbor, N.Y., 1989; Current Protocols in Molecular Biology, Edited by Ausubel F. M. et al., John Wiley and Sons, Inc. New York). Suitable promoters include but are not limited to LTR or SV40 promoter, E. coli lac, tac or trp promoters and the phage lambda P_{L} promoter. Vectors will preferably incorporate an origin of replication as well as selection markers i.e. ampicilin resistance gene. Suitable bacterial vectors include pET, pQE70, pQE60, pQE-9, pD10 phagescript, psiX174, pbluescript SK, pbsks, pNH8A, pNH16a, pNH18A, pNH46A, ptrc99a, pKK223-3, pKK233-3, pDR540, pRIT5 and eukaryotic vectors pBlueBacIII, pWLNEO, pSV2CAT, pOG44, pXT1, pSG, pSVK3, PBPV, pMSG and pSVL. Host cells may be bacterial i.e. E. coli, Bacillus subtilis, Streptomyces; fungal i.e. Aspergillus niger, Aspergillus nidulins; yeast i.e. Saccharomyces or eukaryotic i.e. CHO, COS.

Upon expression of the polypeptide in culture, cells are typically harvested by centrifugation then disrupted~by physical or chemical means (if the expressed polypeptide is not secreted into the media) and the resulting crude extract retained to isolate the polypeptide of interest. Purification of the polypeptide from culture media or lysate may be achieved by established techniques depending on the properties of the polypeptide i.e. using ammonium sulfate or ethanol precipitation, acid extraction, anion or cation exchange chromatography, phosphocellulose chromatography, hydrophobic interaction chromatography, hydroxylapatite chromatography and lectin chromatography. Final purification may be achieved using HPLC.

The polypeptides may be expressed with or without a leader or secretion sequence. In the former case the leader may be removed using post-translational processing (see U.S. Pat. No. 4,431,739; U.S. Pat. No. 4,425,437; and U.S. Pat. No. 4,338,397) or be chemically removed subsequent to purifying the expressed polypeptide.

According to a further aspect, the streptococcal polypeptides of the invention may be used in a diagnostic test for Streptococcus infection, in particular group B Streptococcus infection. Several diagnostic methods are possible, for example detecting Streptococcus organism in a biological sample, the following procedure may be followed:

a) obtaining a biological sample from a host;

b) incubating an antibody or fragment thereof reactive with a polypeptide of the invention with the biological sample to form a mixture; and

c) detecting specifically bound antibody or bound fragment in the mixture which indicates the presence of Streptococcus.

Alternatively, a method for the detection of antibody specific to a Streptococcus antigen in a biological sample containing or suspected of containing said antibody may be performed as follows:

a) obtaining a biological sample from a host;

b) incubating one or more polypeptides of the invention or fragments thereof with the biological sample to form a mixture; and

c) detecting specifically bound antigen or bound fragment in the mixture which indicates the presence of antibody specific to Streptococcus.

One of skill in the art will recognize that this diagnostic test may take several forms, including an immunological test such as an enzyme-linked immunosorbent assay (ELISA), a radioimmunoassay or a latex agglutination assay, essentially to determine whether antibodies specific for the protein are present in an organism. The DNA sequences encoding polypeptides of the invention may also be used to design DNA probes for use in detecting the presence of Streptococcus in a biological sample suspected of containing such bacteria. The detection method of this invention comprises:

a) obtaining the biological sample from a host;

b) incubating one or more DNA probes having a DNA sequence encoding a polypeptide of the invention or fragments thereof with the biological sample to form a mixture; and

c) detecting specifically bound DNA probe in the mixture which indicates the presence of Streptococcus bacteria.

The DNA probes of this invention may also be used for detecting circulating Streptococcus i.e. group B Streptococcus nucleic acids in a sample, for example using a polymerase chain reaction, as a method of diagnosing Streptococcus infections. The probe may be synthesized using conventional techniques and may be immobilized on a solid phase, or may be labelled with a detectable label. A preferred DNA probe for this application is an oligomer having a sequence complementary to at least about 6 contiguous nucleotides of the group B Streptococcus polypeptides of the invention. In a further embodiment, the preferred DNA probe will be an oligomer having a sequence complementary to at least about 15 contiguous nucleotides of the group B Streptococcus polypeptides of the invention. In a further embodiment, the preferred DNA probe will be an oligomer having a sequence complementary to at least about 30 contiguous nucleotides of the group B Streptococcus polypeptides of the invention. In a further embodiment, the preferred DNA probe will be an oligomer having a sequence complementary to at least about 50 contiguous nucleotides of the group B Streptococcus polypeptides of the invention.

Another diagnostic method for the detection of Streptococcus in a host comprises:

a) labelling an antibody reactive with a polypeptide of the invention or fragment thereof with a detectable label;

b) administering the labelled antibody or labelled fragment to the host; and

c) detecting specifically bound labelled antibody or labelled fragment in the host which indicates the presence of Streptococcus.

Alternatively, a method for the detection of antibody specific to a Streptococcus antigen in a biological sample containing or suspected of containing said antibody may be performed as follows:

a) obtaining a biological sample from a host;

b) incubating one or more Streptococcus polypeptides of the invention or fragments thereof with the biological sample to form a mixture; and

c) detecting specifically bound antigen or bound fragment in the mixture which indicates the presence of antibody specific to Streptococcus.

One of skill in the art will recognize that the diagnostic test may take several forms, including an immunological test such as an enzyme-linked immunosorbent assay (ELISA), a radioimmunoassay or a latex agglutination assay, essentially to determine whether antibodies specific for the protein are present in an organism.

The DNA sequences encoding polypeptides of the invention may also be used to design DNA probes for use in detecting the presence of Streptococcus in a biological sample suspected of containing such bacteria. The detection method of this invention comprises:

a) obtaining the biological sample from a host;

b) incubating one or more DNA probes having a DNA sequence encoding a polypeptide of the invention or fragments thereof with the biological sample to form a mixture; and

c) detecting specifically bound DNA probe in the mixture which indicates the presence of Streptococcus bacteria.

According to one aspect, the present invention provides the use of an antibody for treatment and/or prophylaxis of streptococcal infections.

A further aspect of the invention is the use of the Streptococcus polypeptides of the invention as immunogens for the production of specific antibodies for the diagnosis and in particular the treatment of streptococcus infection. Suitable antibodies may be determined using appropriate screening methods, for example by measuring the ability of a particular antibody to passively protect against streptococcus infection in a test model. One example of an animal model is the mouse model described in the examples herein. The antibody may be a whole antibody or an antigen-binding fragment thereof and may belong to any immunoglobulin class. The antibody or fragment may be of animal origin, specifically of mammalian origin and more specifically of murine, rat or human origin. It may be a natural antibody or a fragment thereof, or if desired, a recombinant antibody or antibody fragment. The term recombinant antibody or antibody fragment means antibody or antibody fragment which was produced using molecular biology techniques. The antibody or antibody fragments may be polyclonal, or preferably monoclonal. It may be specific for a number of epitopes associated with the Group B Streptococcus polypeptides but is preferably specific for one.

A further aspect of the invention is the use of the antibodies directed to the polypeptides of the invention for passive immunization. One could use the antibodies described in the present application.

A further aspect of the invention is a method for immunization, whereby an antibody raised by a polypeptide of the invention is administered to a host in an amount sufficient to provide a passive immunization.

In a further embodiment, the invention provides the use of a pharmaceutical composition of the invention in the manufacture of a medicament for the prophylactic or therapeutic treatment of streptococcal infection.

In a further embodiment, the invention provides a kit comprising a polypeptide of the invention for detection or diagnosis of streptococcal infection.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLE 1

This example illustrates the identification of Group B streptococcal BVH-A4 gene. Chromosomal DNA was isolated from different Group B streptococcal strains as previously described (Jayarao B M et al. 1991. J. Clin. Microbiol. 29:2774-2778). A λZAPExpress genomic library was constructed using chromosomal DNA purified from the serotype III Group B streptococcal strain COH1 (Children's Hospital and Medical Center, Seattle, Wash., USA) and screened according to the manufacturer's instructions (Stratagene, La Jolla, Calif.) with a pool of human normal sera. Briefly, the purified chromosomal DNA was partially digested with tsp509I restriction enzyme, and the resulting fragments were electrophoresed on a 1% agarose gel (Bio-Rad). Fragments in the 5- to 10-kb size range were extracted from the gel and ligated to the EcoRI arms of λZAPExpress vector and the vector was encapsidated using the Gigapack II packaging extract (Stratagene). The recombinant phages were used to infect E. coli XL1-Blue MRF′ [Δ(mcrA) 183Δ(mcrCB-hsdSMR-mrr) 173 enda1 supE44 thi-1 recA1 gyrA96 relA1 lac (F′ proAB lacI^{q}ZΔM15 Tn10 [Tet^{R}])], which was then plated onto LB agar. The resulting plaques were lifted onto Hybond-C nitrocellulose membranes (Amersham Pharmacia Biotech, Baie d'Urfée, Canada) pre-impregnated with 10 mM Isopropyl-β-d-thiogalactopyranoside (IPTG: ICN Biomedicals Inc., Costa Mesa, Calif.). The membranes were blocked using phosphate-buffered saline (PBS) with 3% skim milk and were sequentially incubated with the pooled of human sera, peroxydase-labeled goat anti-human immunoglobulins antisera (Jackson Immunoresearch Laboratories Inc., West Grove, Pa.) and substrate. Positive plaques were isolated, purified twice, and the recombinant pBK-CMV plasmids (Stratagene) were excised with the ExAssist helper phage (Stratagene) according to the manufacturer's instructions. Immunoblots using phagemid vectors containing the cloned insert revealed that the pooled human sera reacted with a protein band with an approximate molecular weight of 100 kDa for the clone Al. This clone was then identified as BVH-A4. The sequence of the insert was determined using the TAQ Dye Deoxy Terminator Cycle Sequencing Kit with an Applied Biosystems Inc. (Foster City, Calif.) automated sequencer model 373A according to the manufacturer's recommendations.

### EXAMPLE 2

This example illustrates the cloning of Group B streptococcal BVH-A4 gene.

The coding region of Group B streptococcal BVH-A4 (SEQ ID NO: 1) gene without the region coding for the leader peptide was amplified by PCR (DNA Thermal Cycler GeneAmp PCR system 2400 Perkin Elmer, San Jose, Calif.) from genomic DNA of serotype III Group B streptococcal strain COH1 using oligonucleotide primers that contained base extensions for the addition of restriction sites NcoI (CCATGG) and NotI (GCGGCCGC). The oligonucleotide primers (Table 1) DMAR800 and OCRR588 were used to amplify the BVH-A4 gene. PCR products were purified from agarose gel using a QIAquick gel extraction kit from QIAgen following the manufacturer's instructions (Chatsworth, Calif.), and digested with NcoI and NotI (Pharmacia Canada Inc, Baie d'Urfée, Canada). The pET-21d(+) vector (Novagen, Madison, Wis.) was digested with NcoI and NotI and purified from agarose gel using a QIAquick gel extraction kit from QIAgen (Chatsworth, Calif.). The NcoI-NotI PCR product was ligated to the NcoI-NotI pET-21d(+)expression vector. The ligated product was transformed into E. coli strain DH5α [φ80dlacZΔM15 Δ(lacZYA-argF)U169 endAl recAl hsdR17(r_{K}−m_{K}+) deoR thi-1 supE44 λ⁻ gyrA96 relA1] (Gibco BRL, Gaithersburg, Md.) according to the method of Simanis (Hanahan, D. DNA Cloning, 1985, D. M. Glover (ed), pp. 109-135). Recombinant pET-21d(+)plasmid (rpET21d(+)) containing BVH-A4 gene was purified using a QIAgen plasmid kit (Chatsworth, Calif.) and DNA insert was sequenced (Taq Dye Deoxy Terminator Cycle Sequencing kit, ABI, Foster City, Calif.).

It was determined that the open reading frame (ORF) which codes for BVH-A4 gene (SEQ ID NO: 1) contains 3168-bp and encodes a 1055 amino acid residues polypeptide with a predicted pI of 7.97 and a predicted molecular mass of 118,151.59 Da. Analysis of the predicted amino acid residues sequence (SEQ ID NO:2)using the Spscan software (Wisconsin Sequence Analysis Package; Genetics Computer Group) suggested the existence of a 22 amino acid residues signal peptide (MTKKHLKTLALALTTVSVVTYS), which ends with a cleavage site situated between a serine and a glutamine residues.

TABLE 1

Oligonucleotide primers used for PCR amplifications
of Group B streptococcal BVH-A4 gene

| | Primers | Restriction | | |
|---|---|---|---|---|
| Gene | I.D. | site | Vector | Sequence |
| BVH-A4 | DMAR800 | NcoI | pET21d | 5′- |
| | | | | GCGCCCATGGTGCAGGAGGTATATGGT |
| | | | | TAGAAAG-3′ (SEQ ID No: 3) |
| BVH-A4 | OCRR587 | BamH1 | pET21b | 5′- |
| | | | | GGTGGATCCGAGAAAGGCTTTATTGTA |
| | | | | ATG-3′ (SEQ ID No: 4) |
| BVH-A4 | OCRR588 | NotI | pET21d | 5′- |
| | | | or | CATATTAATTGCGGCCGCTTTTCTTGC |
| | | | pET21b | TCGTTTTCC-3′ (SEQ ID No: 5) |
| BVH-A4 | DMAR752 | BamHI | pCMV-GH | 5′- |
| | | | | CGTTGGATCCTCAGGAGGTATATGGAT |
| | | | | TAGAAAG-3′ (SEQ ID No: 6) |
| BVH-A4 | DMAR753 | SalI | pCMV-GH | 5′- |
| | | | | CATCGTCGACTTATTTTCTTGCTCGTT |
| | | | | TTCC-3′ (SEQ ID No: 7) |

### EXAMPLE 3

This example describes the PCR amplification of Group B streptococcal BVH-A4 gene from other Group B strains

To confirm the presence by PCR amplification of BVH-A4 (SEQ ID NO:1) gene, the following 11 serologically distinct Group B streptococcal strains were used: C388/90 (serotype Ia/c), ATCC12401 (serotype Ib), ATCC27591 (serotype Ic), NCS246 (serotype II/R), NCS954 (serotype III), NCS97SR331 (serotype IV), NCS535 (serotype V), NCS9842 (serotype VI), NCS7271 (serotype VII), NCS970886 (serotype VIII), ATCC27956 (bovine isolate). These strains were obtained from the American Type Culture Collection (Rockville, Md., USA) and National Centre for Streptococcus, Provincial Laboratory of Public Health for Northern Alberta (Edmonton, Canada). The E. coli strain XL1-Blue MRF′ was used in these experiments as negative control. Chromosomal DNA was isolated from each Group B streptococcal strain as previously described (Jayarao BM et al. 1991. J. Clin. Microbiol. 29:2774-2778). BVH-A4 (SEQ ID NO:1) gene was amplified by PCR (DNA Thermal Cycler GeneAmp PCR system 2400 Perkin Elmer, San Jose, Calif.) from the genomic DNA purified from the 11 Group B streptococcal strains, and the control E. coli strain using the oligonucleotides presented in Table 1. The oligonucleotide primers OCRR587,and OCRR588 were used to amplify the BVH-A4 (SEQ ID NO:1) gene. PCR was performed with 35 cycles of 30 sec at 94° C., 30 sec at 55° C. and 150 sec at 72° C. and a final elongation period of 30 min at 72° C. The PCR products were size fractionated in 1% agarose gels and were visualized by ethidium bromide staining. The results of these PCR amplifications are presented in Table 2. The analysis of the amplification products revealed that BVH-A4 (SEQ ID NO:1) gene was present in the genome of all of the 11 Group B streptococcal strains tested. No such product was detected when the control E. coli DNA was submitted to identical PCR amplifications with these oligonucleotide primers.

TABLE 2
Identification of BVH-A4 gene by PCR amplification

| | Strains identification |
|---|---|
| Group B streptococcal isolates | BVH-A4 |
| C388/90 (serotype Ia/c) | + |
| ATCC12401 (serotype Ib) | + |
| ATCC27591 (serotype Ic) | + |
| NCS246 (serotype II/R) | + |
| NCS954 (serotype III) | + |
| NCS97SR331 (serotype IV) | + |
| NCS535 (serotype V) | + |
| NCS9842 (serotype VI) | + |
| NCS7271 (serotype VII) | + |
| NCS970886 (serotype VIII) | + |
| ATCC27956 (bovine isolate) | + |
| E. coli control strain XL1 Blue MRF' | − |

### EXAMPLE 4

This example illustrates the cloning of Group B streptococcal BVH-A4 gene in CMV plasmid PCMV-GH.

The DNA coding region of Group B streptococcal BHV-A4 (SEQ ID NO:1) without the leader peptide was inserted in phase downstream of a human growth hormone (hGH) gene which was under the transcriptional control of the cytomegalovirus (CMV) promotor in the plasmid vector pCMV-GH (Tang et al., Nature, 1992, 356 :152). The CMV promotor is non functional plasmid in E. coli cells but active upon administration of the plasmid in eukaryotic cells. The vector also incorporated the ampicillin resistance gene.

The coding regions of BVH-A4 (SEQ ID NO: 1) gene without its leader peptide regions was amplified by PCR (DNA Thermal Cycler GeneAmp PCR system 2400 Perkin Elmer, San Jose, Calif.) from genomic DNA of serotype III Group B streptococcal strain COH1 using oligonucleotide primers that contained base extensions for the addition of restriction sites BamHI (GGATCC) and SalI (GTCGAC). The oligonucleotide primers DMAR752 and DMAR753 were used to amplify the BVH-A4 (SEQ ID NO:1) gene. The PCR product was purified from agarose gel using a QIAquick gel extraction kit from QIAgen (Chatsworth, Calif.), digested with restriction enzymes (Pharmacia Canada Inc, Baie d′Urfée, Canada). The pCMV-GH vector (Laboratory of Dr. Stephen A. Johnston, Department of Biochemistry, The University of Texas, Dallas, Tex.) was digested with BamHI and SalI and purified from agarose gel using the QIAquick gel extraction kit from QIAgen (Chatsworth, Calif.). The BamHI-SalI DNA fragments were ligated to the BamHI-SalI PCMV-GH vector to create the hGH-BVH-A4 fusion protein under the control of the CMV promoter. The ligated product was transformed into E. coli strain DH5α [φ80dlacZΔM15 Δ(lacZYA-argF)U169 enda1 recA1 hsdR17(r_{K}−m_{K}+) deoR thi-1 supE44 λ⁻gyrA96 relA1] (Gibco BRL, Gaithersburg, Md.) according to the method of Simanis (Hanahan, D. DNA Cloning, 1985, D. M. Glover (ed), pp. 109-135). The recombinant pCMV plasmid was purified using a QIAgen plasmid kit (Chatsworth, Calif.) and the nucleotide sequence of the DNA insert was verified by DNA sequencing.

### EXAMPLE 5

This example illustrates the use of DNA to elicit an immune response to Group B streptococcal BVH-A4 protein antigen. Groups of 8 female BALB/c mice (Charles River, St-Constant, Québec, Canada) were immunized by intramuscular injection of 100 μl three times at two- or three-week intervals with 50 μg of recombinant PCMV-GH encoding BVH-A4 (SEQ ID NO:1) gene in presence of 50 μg of granulocyte-macrophage colony-stimulating factor (GM-CSF)-expressing plasmid pCMV-GH-GM-CSF (Laboratory of Dr. Stephen A. Johnston, Department of Biochemistry, The University of Texas, Dallas, Tex.). As control, groups of mice were injected with 50 μg of pCMV-GH in presence of 50 μg of pCMV-GH-GM-CSF. Blood samples were collected from the orbital sinus prior to each immunization and seven days following the third injection and serum antibody responses were determined by ELISA using purified BVH-A4-His.Tag recombinant proteins as coating antigen.

### EXAMPLE 6

This example illustrates the production and purification of recombinant Group B streptococcal BVH-A4 protein.

The recombinant pET-21d(+)plasmid with BVH-A4 gene corresponding to the SEQ ID NO: 1 was used to transform by electroporation (Gene Pulser II apparatus, BIO-RAD Labs, Mississauga, Canada) E. coli strain BL21(DE3) (F⁻ompT hsdS_{B} (r⁻_{B}m⁻_{B}) gal dcm (DE3)) (Novagen, Madison, Wis.). In this strain of E. coli, the T7 promotor controlling expression of the recombinant protein is specifically recognized by the T7 RNA polymerase (present on the λDE3 prophage) whose gene is under the control of the lac promotor which is inducible by isopropyl-β-d-thio-galactopyranoside (IPTG). The transformants BL21(DE3)/rpET were grown at 37° C. with agitation at 250 rpm in LB broth (peptone log/L, yeast extract 5 g/L, NaCl 10 g/L) containing 100 μg of carbenicillin (Sigma-Aldrich Canada Ltd., Oakville, Canada) per ml until the A₆₀₀ reached a value of 0.6. In order to induce the production of Group B streptococcal BVH-A4-His.Tag recombinant protein, the cells were incubated for 3 additional hours in the presence of IPTG at a final concentration of 1 mM. Induced cells from a 500 ml culture were pelleted by centrifugation and frozen at −70° C.

The purification of the recombinant proteins from the soluble cytoplasmic fraction of IPTG-induced BL21(DE3)/rpET21d(+) was done by affinity chromatography based on the properties of the His.Tag sequence (6 consecutive histidine residues) to bind to divalent cations (Ni²⁺) immobilized on the HiseBind metal chelation resin. Briefly, the pelleted cells obtained from a 500 mL culture induced with IPTG was resuspended in lysis buffer (20 mM Tris, 500 mM NaCl, 10 mM imidazole, pH 7.9) containing 1 mM PMSF, sonicated and centrifuged at 12,000×g for 20 min to remove debris. The supernatant was deposited on a Ni—NTA agarose column (Qiagen, Mississauga, Ontario, Canada). The Group B streptococcal BVH-A4-His.Tag recombinant protein was eluted with 250 mM imidazole-500 mM NaCl-20 mM Tris pH 7.9. The removal of the salt and imidazole from the samples was done by dialysis against PBS at 4° C. The quantities of recombinant proteins obtained from the soluble fraction of E. coli were estimated by MicroBCA (Pierce, Rockford, Ill.).

### EXAMPLE 7

This example illustrates the accessibility to antibodies of the Group B streptococcal BVH-A4 protein at the surface of Group B streptococcal strains.

Bacteria were grown in Todd Hewitt, (TH) broth (Difco Laboratories, Detroit Mich.) with 0.5% Yeast extract (Difco Laboratories) and 0.5% peptone extract (Merck, Darmstadt, Germany) at 37° C. in a 8% CO₂ atmosphere to give an OD₄₉₀nm of 0.600 (˜10⁸ CFU/ml). Dilutions of anti-BVH-A4 or control sera were then added and allowed to bind to the cells, which were incubated for 2 h at 4° C. Samples were washed 4 times in blocking buffer [phosphate-buffered saline (PBS) containing 2% bovine serum albumin (BSA)], and then 1 ml of goat fluorescein (FITC)-conjugated anti-mouse IgG+IgM diluted in blocking buffer was added. After an additional incubation of 60 min at room temperature, samples were washed 4 times in blocking buffer and fixed with 0.25 % formaldehyde in PBS buffer for 18-24 h at 4° C. Cells were washed 2 times in PBS buffer and resuspended in 500 μl of PBS buffer. Cells were kept in the dark at 4° C. until analyzed by flow cytometry (Epics® XL; Beckman Coulter, Inc.).

### EXAMPLE 8

This example illustrates the protection against fatal Group B streptococcal infection induced by passive immunization of mice with rabbit hyper-immune sera.

New Zealand rabbits (Charles River laboratories, St-Constant, Canada) were injected subcutaneously at multiple sites with 50 μg and 100 μg of BVH-A4-His.Tag protein that was produced and purified as described in Example 6 and adsorbed to Alhydrogel adjuvant (Superfos Biosector a/s). Rabbits were immunized three times at three-week intervals with the BVH-A4-His.Tag protein. Blood samples were collected three weeks after the third injection. The antibodies present in the serum were purified by precipitation using 40% saturated ammonium sulfate. Groups of 10 female CD-1 mice (Charles River) were injected intravenously with 500 μl of purified serum collected either from BVH-A4-His.Tag immunized rabbits, or rabbits immunized with an unrelated control recombinant protein. Eighteen hours later the mice were challenged with approximately 8×10⁴ CFU of the Group B streptococcal strain C388/90 (Ia/c). Samples of the Group B streptococcal challenge inoculum were plated on blood agar plates to determine the CFU and to verify the challenge dose. Deaths were recorded for a period of 14 days.

### EXAMPLE 9

This example illustrates the protection of mice against fatal Group B streptococcal infection induced by immunization.

Groups of 8 female CD-1 mice (Charles River) were immunized subcutaneously three times at three-week intervals with 20 μg of either BVH-A4-His.Tag protein that was produced and purified as described in Example 6 in presence of 10 μg of QuilA adjuvant (Cedarlane Laboratories Ltd, Hornby, Canada). The control mice were injected with QuilA adjuvant alone in PBS. Blood samples were collected from the orbital sinus on day 1, 22 and 43 prior to each immunization and seven days (day 50) following the third injection. Two weeks later the mice were challenged with approximately 8×10⁴ CFU of the Group B streptococcal strain C388/90 (Ia/c). Samples of the Group B streptococcal challenge inoculum were plated on blood agar plates to determine the CFU and to verify the challenge dose. Deaths were recorded for a period of 14 days.

1. An isolated polynucleotide comprising a polynucleotide chosen from:
(a) a polynucleotide encoding a polypeptide having at least 80% identity to a second polypeptide comprising a sequence chosen from: SEQ ID NO: 2 or fragments or analogs thereof;
(b) a polynucleotide encoding a polypeptide having at least 95% identity to a second polypeptide comprising a sequence chosen from: SEQ ID NO: 2 or fragments or analogs thereof;
(c) a polynucleotide encoding a polypeptide comprising a sequence chosen from: SEQ ID NO: 2 or fragments or analogs thereof;
(d) a polynucleotide encoding a polypeptide capable of raising antibodies having binding specificity for a polypeptide comprising a sequence chosen from: SEQ ID NO: 2 or fragments or analogs thereof;
(e) a polynucleotide encoding an epitope bearing portion of a polypeptide comprising a sequence chosen from SEQ ID NO: 2 or fragments or analogs thereof;
(f) a polynucleotide comprising a sequence chosen from SEQ ID NO: 1 or fragments or analogs thereof;
(g) a polynucleotide that is complementary to a polynucleotide in (a), (b), (c), (d), (e) or (f).

2. An isolated polynucleotide comprising a polynucleotide chosen from:
(a) a polynucleotide encoding a polypeptide having at least 80% identity to a second polypeptide comprising a sequence chosen from: SEQ ID NO: 2;
(b) a polynucleotide encoding a polypeptide having at least 95% identity to a second polypeptide comprising a sequence chosen from: SEQ ID NO: 2;
(c) a polynucleotide encoding a polypeptide comprising a sequence chosen from: SEQ ID NO: 2;
(d) a polynucleotide encoding a polypeptide capable of raising antibodies having binding specificity for a polypeptide comprising a sequence chosen from: SEQ ID NO: 2;
(e) a polynucleotide encoding an epitope bearing portion of a polypeptide comprising a sequence chosen from SEQ ID NO: 2;
(f) a polynucleotide comprising a sequence chosen from SEQ ID NO: 1;
(g) a polynucleotide that is complementary to a polynucleotide in (a), (b), (c), (d), (e) or (f).

3. The polynucleotide of claim 1, wherein said polynucleotide is DNA.

4. The polynucleotide of claim 2, wherein said polynucleotide is DNA.

5. The polynucleotide of claim 1, wherein said polynucleotide is RNA.

6. The polynucleotide of claim 2, wherein said polynucleotide is RNA.

7. An isolated polynucleotide that hybridizes under stringent conditions to either
(a) a DNA sequence encoding a polypeptide or
(b) the complement of a DNA sequence encoding a polypeptide;
wherein said polypeptide comprises SEQ ID NO:2 or fragments or analogs thereof.

8. The polynucleotide of claim 1 that hybridizes under stringent conditions to either
(a) a DNA sequence encoding a polypeptide or
(b) the complement of a DNA sequence encoding a polypeptide;
wherein said polypeptide comprises SEQ ID NO: 2, or fragments or analogs thereof.

9. The polynucleotide of claim 2 that hybridizes under stringent conditions to either
(a) a DNA sequence encoding a polypeptide or
(b) the complement of a DNA sequence encoding a polypeptide;
wherein said polypeptide comprises SEQ ID NO: 2.

10. The polynucleotide of claim 1 that hybridizes under stringent conditions to either
(a) a DNA sequence encoding a polypeptide or
(b) the complement of a DNA sequence encoding a polypeptide;
wherein said polypeptide comprises at least 10 contiguous amino acid residues from a polypeptide comprising SEQ ID NO: 2, or fragments or analogs thereof.

11. The polynucleotide of claim 2 that hybridizes under stringent conditions to either
(a) a DNA sequence encoding a polypeptide or
(b) the complement of a DNA sequence encoding a polypeptide;
wherein said polypeptide comprises at least 10 contiguous amino acid residues from a polypeptide comprising SEQ ID NO: 2.

12. A vector comprising the polynucleotide of claim 1, wherein said DNA is operably linked to an expression control region.

13. A vector comprising the polynucleotide of claim 2, wherein said DNA is operably linked to an expression control region.

14. A host cell transfected with the vector of claim 12.

15. A host cell transfected with the vector of claim 13.

16. A process for producing a polypeptide comprising culturing a host cell according to claim 14 under conditions suitable for expression of said polypeptide.

17. A process for producing a polypeptide comprising culturing a host cell according to claim 15 under condition suitable for expression of said polypeptide.

18. An isolated polypeptide comprising a polypeptide chosen from:
(a) a polypeptide having at least 80% identity to a second polypeptide having an amino acid sequence comprising: SEQ ID NO: 2, or fragments or analogs thereof;
(b) a polypeptide having at least 95% identity to a second polypeptide having an amino acid sequence comprising: SEQ ID NO: 2, or fragments or analogs thereof;
(c) a polypeptide comprising a sequence chosen from SEQ ID NO: 2, or fragments or analogs thereof;
(d) a polypeptide capable of raising antibodies having binding specificity for a polypeptide comprising SEQ ID NO: 2, or fragments or analogs thereof;
(e) an epitope bearing portion of a polypeptide comprising SEQ ID NO: 2, or fragments or analogs thereof;
(f) the polypeptide of (a), (b), (c), (d), or (e) wherein the N-terminal Met residue is deleted;
(g) the polypeptide of (a), (b), (c), (d), (e), or (f) wherein the secretory amino acid sequence is deleted.

19. An isolated polypeptide comprising a polypeptide chosen from:
(a) a polypeptide having at least 80% identity to a second polypeptide having an amino acid sequence comprising: SEQ ID NO: 2;
(b) a polypeptide having at least 95% identity to a second polypeptide having an amino acid sequence comprising: SEQ ID NO: 2;
(c) a polypeptide comprising a sequence chosen from SEQ ID NO: 2;
(d) a polypeptide capable of raising antibodies having binding specificity for a polypeptide comprising SEQ ID NO: 2;
(e) an epitope bearing portion of a polypeptide comprising SEQ ID NO: 2;
(f) the polypeptide of (a), (b), (c), (d), or (e) wherein the N-terminal Met residue is deleted;
(g) the polypeptide of (a), (b), (c), (d), (e), or (f) wherein the secretory amino acid sequence is deleted.

20. A chimeric polypeptide comprising two or more polypeptides having a sequence chosen from SEQ ID NO: 2, or fragments or analogs thereof; provided that the polypeptides are linked as to formed a chimeric polypeptide.

21. A chimeric polypeptide comprising two or more polypeptides having a sequence chosen from SEQ ID NO: 2; provided that the polypeptides are linked as to formed a chimeric polypeptide.

22. A pharmaceutical composition comprising a polypeptide according to any one of claims 18 to 21 and a pharmaceutically acceptable carrier, diluent or adjuvant.

23. A method for prophylactic or therapeutic treatment of GBS bacterial infection in a host susceptible to GBS infection comprising administering to said host a prophylactic or therapeutic amount of a composition according to claim 22.

24. A method according to claim 23 wherein the host is a neonate or an infant.

25. A method according to claim 24 wherein said infection causes sepsis, meningitis, pneumonia, cellulitis, osteomyelitis, septic arthritis, endocarditis or epiglottis.

26. A method according to claim 23 wherein the host is a pregnant woman.

27. A method according to claim 26 wherein said infection causes mild urinary tract infection to life-threatening sepsis and meningitis, including also osteomyelitis, endocarditis, amniotis, endometritis, wound infections (postcesarean and postepisiotomy), cellulitis or fasciitis.

28. A method according to claim 23 wherein the host is a non-pregnant adult.

29. A method according to claim 28 wherein said infection causes primary bacteremia, skin or soft tissue infection, pneumonia, urosepsis, endocarditis, peritonitis, meningitis or empyema.

30. A method according to claim 23 wherein the host is a member of a dairy herd.

31. A method according to claim 30 wherein said infection causes mastitis.

32. A method for diagnostic of GBS bacterial infection in an host susceptible to GBS infection comprising
(a) obtaining a biological sample from a host;
(b) incubating an antibody or fragment thereof reactive with a polypeptide according to any one of claims 18 to 21 with the biological sample to form a mixture; and
(c) detecting specifically bound antibody or bound fragment in the mixture which indicates the presence of Streptococcus.

33. A method for diagnostic of GBS bacterial infection in an host susceptible to GBS infection comprising
(a) obtaining a biological sample from a host;
(b) incubating one or more polypeptides according to any one of claims 18 to 21 or fragments thereof with the biological sample to form a mixture; and
(c) detecting specifically bound antigen or bound fragment in the mixture which indicates the presence of antibody specific to Streptococcus.

34. Use of the pharmaceutical composition according to claim 22 in the manufacture of a medicament for the prophylactic or therapeutic treatment of streptococcal infection.

35. Kit comprising a polypeptide according to any one of claims 18 to 21 for detection or diagnosis of streptococcal infection.
